(19) United States
(12) Patent Application Publication
(10) Pub. No.: US 2021/0393197 A1
(43) Pub. Date: Dec. 23, 2021

(54) Pharmacodynamic Model for Determining Last Use of Inhaled and Oral Cannabis Products

(71) Applicant: ImmunoTess, Inc., Roseville, CA (US)

(72) Inventors: Michael W DeGregorio, Granite Bay, CA (US); Chiao-Jung Kao, Granite Bay, CA (US); Gregory T Wurz, Lincoln, CA (US)

(73) Assignee: RCU Labs, Inc., Roseville, CA (US)

(21) Appl. No.: 17/048,737

(22) PCT Filed: Apr. 19, 2019
(86) PCT No.: PCT/US2019/028298

§ 371 Date: Oct. 19, 2020

Related U.S. Application Data
(60) Provisional application No. 62/661,280, filed on Apr. 23, 2018.

(51) Int. Cl. A61B 5/00; A61B 5/15; A61B 5/151; G01N 33/94

(52) U.S. Cl. CPC A61B 5/4845; A61B 5/150022; A61B 2503/20; G01N 33/948; A61B 5/15142

(57) ABSTRACT

The present invention provides a method for determining recent use of cannabis in human subjects, the method comprising collecting samples of whole blood separated in time utilizing a device that collects and stores capillary blood for LC-MS/MS analysis. In particular embodiments, the method is used by law enforcement personnel to collect evidence in driving under the influence investigations. In some embodiments, the method is utilized by employers in routine monitoring of workplace drug policy compliance among employees and in workplace accident investigations.

### CROSS-REFERENCES TO RELATED APPLICATIONS

This application claims priority to U.S. Provisional Patent Application No. 62/661,280, filed Apr. 23, 2018, the disclosure of which is hereby incorporated by reference in its entirety for all purposes.

### BACKGROUND OF THE INVENTION

Cannabis (Cannabis spp) remains the most commonly abused illicit drug in the United States and throughout the world [United Nations Office on Drugs and Crime, World Drug Report (2017)]. This complex plant, used by human beings for thousands of years, contains over 100 cannabinoids, also called phytocannabinoids, as well as hundreds of other chemical compounds such as terpenes and flavonoids [ElSohly M A. et al., Prog Chem Org Nat Prod 103:1-36 (2017)]. The primary psychoactive constituent of cannabis is Δ⁹-tetrahydrocannabinol (Δ⁹-THC), which exerts its activity by binding to endogenous cannabinoid receptors known as CB₁ and CB₂ [Maccarrone M. et al., Trends Pharmacol Sci 36:277-96 (2015)]. Other common cannabinoids found in cannabis include cannabidiol (CBD), cannabigerol (CBG), cannabinol (CBN), cannabichromene (CBC), and Δ⁹-tetrahydrocannabivarin (Δ⁹-THCV). Within the cannabis plant, CBD, CBG, Δ⁹-THCV and Δ⁹-THC exist largely in their carboxylic acid forms, which undergo decarboxylation when heated. In order to become psychoactive, Δ⁹-THC acid requires decarboxylation [Grotenhermen F., Clin Pharmacokinet 42:327-60 (2003)].

In the United States, cannabis has been approved for medicinal use in a total of 33 states, with recreational use now legal in a total of 10 states and the District of Columbia as of early 2019. Driving under the influence (DUI) of drugs has become a critical public safety issue, and will become only more so with the expanding legalization of cannabis for medicinal and recreational use. According to data from the National Highway Traffic Safety Administration, the number of drivers testing positive for cannabis who were killed in crashes in the United States doubled between 2007 and 2015 [Berning A. et al., National Highway Traffic Safety Administration Report DOT HS 812 118 (2015)]. In May 2016, the AAA Foundation for Traffic Safety published a study entitled “Prevalence of Marijuana Involvement in Fatal Crashes: Washington, 2010-2014,” the results of which showed that the number of drivers involved in fatal crashes in the State of Washington who tested positive for Δ⁹-THC more than doubled, from approximately 8.3% in 2013 to 17.0% in 2014 [Tefft B C. et al., AAA Foundation for Traffic Safety (2016)]. However, these results do not show whether these drivers were actually impaired by Δ⁹-THC at the time of the crash. The State of Washington legalized recreational cannabis in November 2012. The State of Colorado, where recreational cannabis was also legalized following the November 2012 election, experienced a sharp increase of approximately 153% (from 55 to 139) in the years 2013 to 2017 in the number of drivers involved in fatal crashes who tested positive for marijuana use [Reed J K., Impacts of Marijuana Legalization in Colorado, Colorado Department of Public Safety (October 2018)].

Cannabis is the most commonly detected illicit drug in drivers [Berning A. et al., National Highway Traffic Safety Administration Report DOT HS 812 118 (2015); Legrand S A. et al., Drug Test Anal 5:156-165 (2013)]; unfortunately, however, there is still no reliable method of proving recent cannabis use in DUI investigations. While some states, e.g., Colorado and Washington, have enacted per se limits for driving under the influence of cannabis, no definitive correlation between the degree of impairment and specific blood levels of Δ⁹-THC has been established [Logan B. et al., An Evaluation of Data from Drivers Arrested for Driving Under the Influence in Relation to Per se Limits for Cannabis, AAA Foundation for Traffic Safety (May 2016)]. The State of California and other U.S. states where cannabis has been legalized presently rely on specially-trained police officers known as drug recognition experts to make a determination of DUI due to cannabis or other drugs. Law enforcement agencies desperately need an objective means of assessing cannabis impairment to support their initial determinations of driver intoxication.

Unlike alcohol (ethanol), the elimination of which follows zero order kinetics [Jones A W., Forensic Sci Int 200:1-20 (2010)], the pharmacokinetics of cannabis-derived cannabinoids are complex and can vary depending on the route of administration [Huestis M A., In: Handbook of Experimental Pharmacology, Stark K. (ed.) 168:657-90 (2005)]. This complicates the assessment of impairment in DUI investigations, where time of last use needs to be estimated as accurately as possible. The window of peak impairment due to ingestion of Δ⁹-THC through smoking, vaping or oral consumption is generally agreed to last approximately 4-5 hours after administration, with oral consumption lagging about an hour behind inhalation routes [Huestis M A. et al., Clin Chem 51:2289-95 (2005); Couper F. et al., In: Drugs and Human Performance Fact Sheets, National Highway Traffic Safety Administration Report DOT HS 809 725:7-12 (2004); Vandrey R. et al., J Anal Toxicol 41:83-99 (2017)]. Δ⁹-THC is a highly lipophilic compound that rapidly distributes into tissues following absorption (see FIG. 2). The distribution half-life of Δ⁹-THC has been estimated to be between 24 and 55 minutes [Moeller M R. et al., J Forensic Sci 37:969-83 (1992); Wall M E. et al., Clin Pharmacol Ther 34:352-63 (1983)]. However, blood samples are typically not collected until approximately 1.5-4 hours after a traffic stop or collision [Rohrig T P. et al., Drug Test Anal 10:663-70 (2018); Jones A W., Addiction 103:452-61 (2008); Biecheler M B. et al., Traffic Inj Prev 9:11-21 (2008)], a time when plasma Δ⁹-THC concentrations may have already fallen to very low levels (see FIG. 2).

In humans, the main intermediate metabolite of Δ⁹-THC is 11-hydroxy-Δ⁹-THC (11-OH-Δ⁹-THC), which is produced primarily in the liver by cytochrome P450 (CYP) 2C9, 2C19 and 3A4 isozymes [Matsunaga T. et al., Life Sci 56:2089-95 (1995)] (see FIG. 1). 11-OH-Δ⁹-THC is also the most psychoactive of the Δ⁹-THC metabolites, and has been shown to be equipotent to Δ⁹-THC in humans [Perez-Reyes M. et al., Science 177:633-5 (1972)] and even more potent in some animal models [Karler R. et al., NIDA Res Monogr 79:96-107 (1987)]. Subsequently, 11-OH-Δ⁹-THC can be eliminated in the feces or oxidized to the inactive metabolite 11-nor-9-carboxy-Δ⁹-THC (commonly referred to as Δ⁹-THC-COOH), which is then conjugated with glucuronic acid in phase II and eliminated in the urine [Grotenhermen F., Clin Pharmacokinet 42:327-60 (2003)]. Although glucuronidation increases polarity and thus water solubility, renal clearance of these metabolites is still relatively low due to extensive protein binding [Huestis M A., In: Handbook of Experimental Pharmacology, Stark K. (ed.) 168:657-90 (2005)]. Less common metabolites are formed by side chain hydroxylation at the 1′, 2′, 3′ or 4′ position, 8α- and 8β-hydroxylation through CYP3A4 and 9β,10β- and 9α,10α-epoxidation, also through CYP3A4 [Dinis-Oliveira R J., Drug Metab Rev 48:80-7 (2016)] (see FIG. 1). Interestingly, these epoxides have been shown to be resistant to hydrolysis by epoxide hydrolase [Yamamoto I. et al., J Pharmacobiodyn 7:254-62 (1984)]. Glucuronidation of Δ⁹-THC at the phenolic C1 position is also observed, in addition to multiple other minor metabolic pathways [Dinis-Oliveira R J., Drug Metab Rev 48:80-7 (2016)].

Key cannabis recent use indicators include CBG, CBN and Δ⁹-THCV in plasma, and Δ⁹-THC-glucuronide in plasma and urine [Huestis M A. et al., Trends Mol Med 24:156-72 (2018)]. In plasma, Δ⁹-THC-glucuronide and CBN are not detectable beyond two hours after smoking even in frequent users [Newmeyer M N. et al., Clin Chem 62:1579-92 (2016); Schwope D M. et al., Clin Chem 57:1406-14 (2011)], making them excellent recent use markers. Cannbigerol has also been shown to be an indicator of recent use, being detectable in both frequent and occasional smokers for up to 16 minutes following inhalation [Newmeyer M N. et al., Clin Chem 62:1579-92 (2016)]. Relatively poor detectability in plasma has been noted for Δ⁹-THCV, but it is still a valuable marker of recent use if detected [Newmeyer M N. et al., Clin Chem 62:1579-92 (2016)]. Notably, CBN, CBG and Δ⁹-THCV have not been observed at quantifiable levels in plasma following oral consumption of cannabis [Newmeyer M N. et al., Clin Chem 62:1579-92 (2016)]. In urine, the creatinine-normalized Δ⁹-THC-glucuronide concentration indicates use within six hours of the first sample, with a probability of approximately 93%, if there is greater than a 50% difference in concentration between consecutive samples (drawn within eight hours), and the first sample contains ≥2 μg/g Δ⁹-THC-glucuronide [Desrosiers N A. et al., Clin Chem 60:361-72 (2014)]. The presence of 8β,11-dihydroxy-Δ⁹-THC in urine and/or Δ⁹-THC epoxides may also be potential indicators of recent cannabis use. Urinary concentrations of 15-20 ng/mL of 8β,11-dihydroxy-Δ⁹-THC have been shown to indicate recent cannabis use within 4-6 hours [McBurney L J. et al., J Anal Toxicol 10:56-64 (1986)], although this marker has not seen widespread application [Huestis M A., In: Handbook of Experimental Pharmacology, Stark K. (ed.) 168:657-90 (2005)]. Because of their resistance to hydrolysis by epoxide hydrolase [Yamamoto I. et al., J Pharmacobiodyn 7:254-62 (1984)], the epoxides of Δ⁹-THC may be detectable by modern instrumentation within a short time period following cannabis use.

Pharmacokinetic data for Δ⁹-THC and its metabolites also yield valuable information in regard to recent cannabis use. For example, following smoking, Δ⁹-THC is rapidly absorbed and distributed, with a half-life ranging from approximately 24-55 minutes [Moeller M R. et al., J Forensic Sci 37:969-83 (1992); Wall M E. et al., Clin Pharmacol Ther 34:352-63 (1983)]. If consecutive blood samples are drawn within 30 minutes, and the second sample shows a Δ⁹-THC concentration≤50% compared to the first sample, this would indicate that Δ⁹-THC is still in the distribution phase, strongly suggesting very recent use by smoking, especially when accompanied by an increasing Δ⁹-THC-COOH/Δ⁹-THC concentration ratio. Should the samples show increasing levels of Δ⁹-THC coupled with similar concentrations of 11-OH-Δ⁹-THC and higher concentrations of Δ⁹-THC-COOH, this is strongly suggestive of recent oral consumption of cannabis. Concentrations of both 11-OH-Δ⁹-THC and Δ⁹-THC-COOH have been shown to be much higher following oral administration of Δ⁹-THC compared to inhalation [Huestis M A., In: Handbook of Experimental Pharmacology, Stark K. (ed.) 168:657-90 (2005); Wall M E. et al., Clin Pharmacol Ther 34:352-63 (1983)]. The peak psychoactive effects of Δ⁹-THC are experienced when the concentrations of Δ⁹-THC-COOH and Δ⁹-THC are approximately equal, which occurs within 30-45 minutes after smoking [Huestis M A., In: Handbook of Experimental Pharmacology, Stark K. (ed.) 168:657-90 (2005); Mason A P. et al., J Forensic Sci 30:615-31 (1985); Huestis M A. et al., J Anal Toxicol 16:276-82 (1992); Kelly P. et al., J Anal Toxicol 16:228-35 (1992)]. Finally, a Δ⁹-THC plasma concentration≥5 ng/mL combined with a Δ⁹-THC-COOH/11-OH-Δ⁹-THC concentration ratio<20 indicates the use of cannabis within the last eight hours in frequent smokers regardless of the route of administration. Applying the same formula to occasional smokers indicates cannabis use within 90 minutes by inhalation (smoking/vaping) or oral consumption within the last 12 hours [Newmeyer M N. et al., Clin Chem 62:1579-92 (2016)].

Oral fluid represents a convenient, non-invasive alternative matrix to blood and urine for the assessment of recent cannabis intake in multiple settings; for example, DUI investigations, workplace drug policy compliance and drug treatment. In fact, on-site oral fluid screening devices are already being used by law enforcement in Europe [Newmeyer M N. et al., Clin Chem 63:647-62 (2017)] and are being tested in the United States. An oral fluid Δ⁹-THC concentration≥2 ng/mL indicates recent cannabis use within the last 24 hours, regardless of cannabis usage history [Desrosiers N A. et al., Anal Bioanal Chem 406:4117-28 (2014)]. Other cannabinoid markers of recent cannabis use include Δ⁹-THCV, which can be detected for up to eight hours in occasional users and 12 hours in frequent cannabis users, and CBG, which is detectable up to 26 hours in occasional and 20 hours in frequent cannabis users [Swortwood M J. et al., Drug Test Anal 9:905-15 (2017)]. The presence of the Δ⁹-THC-COOH metabolite in oral fluid can be used as a good general indicator of cannabis use because it can differentiate between passive environmental exposure and actual cannabis consumption, it can identify oral cannabis consumption, and it can be detected for up to three days following last use [Milman G. et al., Clin Chem 56:1261-9 (2010); Lee D. et al., Clin Chem 58:748-56 (2012)].

The general pharmacokinetic concepts described above can also be applied to non-DUI investigations related to cannabis use as well as for DUI investigations related to drugs other than cannabis. For example, cannabis use within the last 24 hours is indicated by a Δ⁹-THC/11-OH-Δ⁹-THC concentration ratio>1 combined with a Δ⁹-THC-COOH/11-OH-Δ⁹-THC concentration ratio<15 regardless of the route of administration or frequency of cannabis use [Newmeyer M N. et al., Clin Chem 62:1579-92 (2016)]. While this approach would not be applicable in cases of determining driver impairment, it would fit nicely in a drug monitoring or compliance setting because it can be used to discriminate between recent use (within the last day) and prior use regardless of smoking status.

Another factor to consider when trying to assess cannabis impairment is the potential for drug-drug interactions between cannabinoids and prescription and non-prescription drugs due to shared metabolic pathways and pharmacologic activity. As previously mentioned, Δ⁹-THC is primarily metabolized by cytochrome P450 3A and 2C enzyme subfamilies [Zendulka O. et al., Curr Drug Metab 17:206-26 (2016)], which are involved in the metabolism of a majority of prescription and non-prescription drugs. While ethanol, which is commonly consumed in combination with cannabis, does not appear to have any meaningful effects on the metabolism of Δ⁹-THC, it does increase peak blood levels and intensify the sedative effects of Δ⁹-THC [Pryor G T. et al., Pharmacol Biochem Behav 7:331-45 (1977); Hollister L E., NIDA Res Monogr 68:110-6 (1986); Hartman R L. et al., Clin Chem 61:850-69 (2015)]. Cannabidiol has been identified as a potent inhibitor of CYP3A4 and CYP2C19 [Jiang R. et al., Drug Metab Pharmacokinet 28:332-8 (2013); Yamaori S. et al., Life Sci 88:730-6 (2011)], and may potentiate the psychoactive effects of Δ⁹-THC [Klein C. et al., Psychopharmacol 218:443-57 (2011)]. Drug interactions between cannabis and benzodiazepines [Pryor G T. et al., Pharmacol Biochem Behav 7:331-45 (1977); Jeffrey A L. et al., Epilepsia 56:1246-51 (2015)], barbiturates [Pryor G T. et al., Pharmacol Biochem Behav 7:331-45 (1977); Seamon M J. et al., Am J Health Syst Pharm 64:1037-44 (2007)], antifungals [Stout S M. et al., Drug Metab Rev 46:86-95 (2014)], tricyclic anti-depressants and selective serotonin reuptake inhibitors [Seamon M J. et al., Am J Health Syst Pharm 64:1037-44 (2007)], and many other drug classes [Alsherbiny M A. et al., Medicines 6:3 (2018)], have been described, leading to potential intensification of Δ⁹-THC psychoactivity.

Given the increasing acceptance of cannabis for both medicinal and recreational use in the United States and internationally, and the current lack of an effective method of discriminating recent use of cannabis from past use, there is a need in the art for methods of determining recent use of cannabis so that impaired individuals can be more accurately identified without penalizing those who test positive for prior cannabis use but are not impaired.

### BRIEF SUMMARY OF THE INVENTION

In some aspects, the present invention provides a method for determining recent use of cannabis in human subjects, the method comprising collecting two or more samples of whole blood separated in time by approximately 20 to 30 minutes utilizing a device that automatically collects and stores capillary blood for later laboratory analysis. The results of the laboratory analysis are used to compute the probability, based on a statistical model, that a subject has recently used cannabis by smoking, vaping, consumption of edibles or other route of administration. The model is based on six or more parameters that have been associated with the recent use of cannabis. The higher the number of parameters for which a subject is positive, the higher the probability of recent use.

In particular aspects, the method is used by law enforcement personnel to collect evidence in driving under the influence investigations, the method comprising the collection of two or more whole blood samples approximately 30 minutes apart using a device that automatically collects and stores capillary blood for later analysis. The samples are then subjected to laboratory analysis for the determination of Δ⁹-THC, Δ⁹-THC metabolites and other cannabinoids. Following the analysis, the data are compared to a list of six pharmacokinetic parameters associated with recent use of cannabis. A positive result in three of the six parameters indicates a 95% probability of recent use. A positive result in four or more of the six parameters indicates a 99% probability of recent use of cannabis. Additional parameters may be used as they are identified to strengthen the statistical power of the model.

In some embodiments, the method is used by employers in the routine monitoring of workplace drug policy compliance among employees and in workplace accident investigations, the method comprising the collection of two or more whole blood samples, which are then subjected to laboratory analysis for the determination of Δ⁹-THC, Δ⁹-THC metabolites and other cannabinoids. Other sample types, including oral fluid and urine, may be collected. Following the analysis, the data are compared to a list of six pharmacokinetic parameters associated with recent use of cannabis. A positive result in three of the six parameters indicates a 95% probability of recent use. A positive result in four or more of the six parameters indicates a 99% probability of recent use of cannabis. Additional parameters may be used as they are identified to strengthen the statistical power of the model.

In particular embodiments, the employers collect whole blood samples from employees using either a device that automatically collects and stores capillary blood for later laboratory analysis, or a standard lancing device similar to that used by diabetics for routine blood glucose monitoring.

In some embodiments, the method is used by law enforcement personnel to collect evidence in DUI investigations involving drugs other than cannabis, the method comprising the collection of two or more whole blood samples approximately 20 to 30 minutes apart utilizing an automatic collection device as described. The samples are then subjected to laboratory analysis for the determination of key drug compounds and metabolites of interest. Following the analysis, the data are compared to a list of six or more pharmacokinetic parameters associated with the recent use of the compound(s) of interest. A positive result in three of the six parameters indicates a 95% probability of recent use. A positive result in four or more of the six parameters indicates a 99% probability of recent use of the compound(s) of interest. Additional parameters may be used as they are identified to strengthen the statistical power of the model.

In particular embodiments, the method is used by law enforcement personnel to collect evidence in DUI investigations involving cannabis combined with prescription or non-prescription drugs, the method comprising the collection of two or more whole blood samples approximately 20 to 30 minutes apart utilizing an automatic collection device as described. The samples are then subjected to laboratory analysis for the determination of Δ⁹-THC, Δ⁹-THC metabolites, other cannabinoids, and other drugs that are known to interact with cannabis, such that the psychoactive effects of cannabis may be prolonged.

In some embodiments, the method is used by employers in the routine monitoring of workplace drug policy compliance among employees and in workplace accident investigations, the method comprising the collection of two or more whole blood samples, which are then subjected to laboratory analysis for cannabis and other drugs that are known to interact with cannabis such that the psychoactive effects of cannabis may be prolonged. Following the analysis, the data are compared to a list of six pharmacokinetic parameters associated with the recent use of the compound(s) of interest. A positive result in three of the six parameters indicates a 95% probability of recent use. A positive result in four or more of the six parameters indicates a 99% probability of recent use of cannabis. Additional parameters may be used as they are identified to strengthen the statistical power of the model.

In particular embodiments, the method is used by employers in the routine monitoring of workplace drug policy compliance among employees and in workplace accident investigations, the method comprising the collection of two or more whole blood samples, which are then subjected to laboratory analysis for drugs of abuse other than cannabis. Other sample types, including oral fluid and urine, may be collected. Following the analysis, the data are compared to a list of six pharmacokinetic parameters associated with the recent use of the compound(s) of interest. A positive result in three of the six parameters indicates a 95% probability of recent use. A positive result in four or more of the six parameters indicates a 99% probability of recent use of the compound(s) of interest. Additional parameters may be used as they are identified to strengthen the statistical power of the model.

Other objects, features, and advantages of the present invention will be apparent to one of skill in the art from the following detailed description and figures.

### BRIEF DESCRIPTION OF THE DRAWINGS

FIG. 1. Metabolism of Δ⁹-THC. The major metabolites of Δ⁹-THC and the metabolic enzymes primarily responsible for their formation are shown. CYP=cytochrome P450; UGT=uridine 5′-diphospho-glucuronosyltransferase; gluc=glucuronic acid.

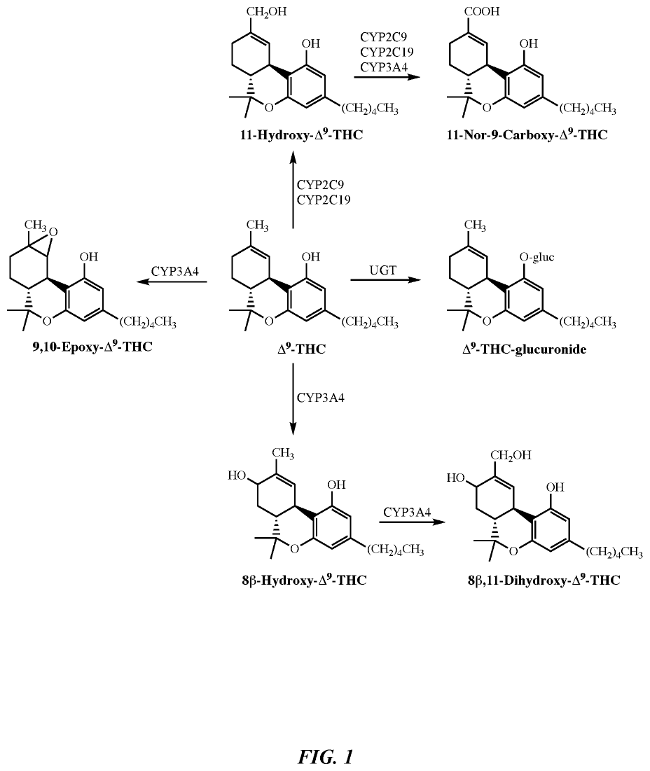

FIG. 2. Typical Δ⁹-THC blood concentration profile after smoking in a recent cannabis user compared to a non-recent user.

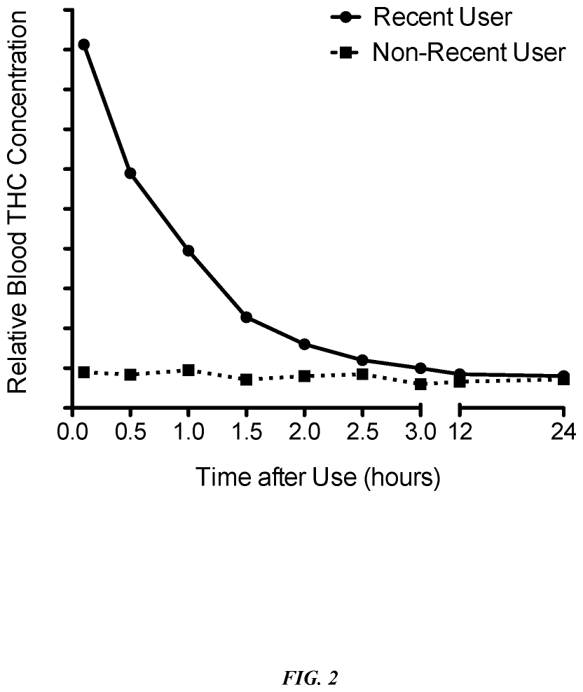

FIG. 3. Clinical data summary: immediately after smoking to 20 minutes post-smoking. Following the analysis of whole blood samples for Δ⁹-THC and metabolites from 14 subjects given a 500-mg cannabis cigarette to smoke, pharmacokinetic data (0-20 minutes post-smoking) was subjected to a six-parameter recent use model and compared to the results from samples collected from 11 non-recent users over the same time interval. *p<0.05, **p<0.01.

FIG. 4. Clinical data summary: 120 minutes to 140 minutes post-smoking. Following the analysis of whole blood samples for Δ⁹-THC and metabolites from six subjects given a 500-mg cannabis cigarette to smoke, pharmacokinetic data (120-140 minutes post-smoking) was subjected to a six-parameter recent use model and compared to the results from samples collected from 11 non-recent users over the same time interval. *p<0.01

FIG. 5. Δ⁹-THC metabolite conversion. The conversion of Δ⁹-THC metabolites 11-OH-Δ⁹-THC to 11-nor-9-carboxy-Δ⁹-THC and 8β-hydroxy-Δ⁹-THC to 8β,11-dihydroxy-Δ⁹-THC in a recent cannabis user, after smoking a 500-mg cannabis cigarette (black bars), compared to a non-recent smoker (gray bars) are shown.

FIG. 6. Clinical data summary: 180 minutes to 200 minutes post-smoking. Following the analysis of whole blood samples for Δ⁹-THC and metabolites from six subjects given a 500-mg cannabis cigarette to smoke, pharmacokinetic data (180-200 minutes post-smoking) was subjected to a six-parameter recent use model and compared to the results from samples collected from 11 non-recent users over the same time interval. *p<0.05, **p<0.01.

FIG. 7. Cannabinoid and metabolite detection in non-recent users. The profile of detection of five different cannabinoids and Δ⁹-THC metabolites (Δ⁹-THC, CBN, 11-OH-Δ⁹-THC, Δ⁹-THC-COOH, 8β,11-dihydroxy-Δ⁹-THC) in 11 non-recent cannabis users is shown. ND=no detection.

FIG. 8. Confidence of predicting recent use. The percentages of subjects at each of three different time intervals after smoking a 500-mg cannabis cigarette for whom the described model had a recent use prediction confidence level of 95% (grey bars) and 99% (checkered bars) are shown.

FIG. 9. Cannabis smoking versus edibles: a case study. Positive recent use parameters following different time intervals after consumption through smoking (500-mg cannabis cigarette) or ingestion of a cannabis edible (30 mg Δ⁹-THC) in the same subject are shown.

### DETAILED DESCRIPTION OF THE INVENTION

I. Introduction

This invention involves the development of a model and test for determining the time of last cannabis use based on the presence of key recent use indicators, including but not limited to CBN, CBG, Δ⁹-THCV, Δ⁹-THC-epoxides and Δ⁹-THC-glucuronide, pharmacokinetic information for Δ⁹-THC and its principal metabolites (11-OH-Δ⁹-THC, Δ⁹-THC-COOH, and 8β,11-dihydroxy-Δ⁹-THC), and concentration ratios of Δ⁹-THC to these metabolites, including the ratios between different metabolites. For the evaluation of recent cannabis use in a DUI investigation, blood specimens are collected on-site from a suspect who, in the judgment of law enforcement, after performing standard roadside sobriety testing, is impaired. The blood samples are collected using a device designed to collect capillary blood automatically into a receptacle containing appropriate anticoagulant additives so as to provide sufficient material to provide both a primary sample and a duplicate. At least two (2) samples are collected to provide the necessary pharmacokinetic information. The second blood sample is ideally collected approximately 20 to 30 minutes after collection of the first sample. The blood collection device operates in a fashion similar to lancing devices used by diabetics to collect blood for glucose testing; that is, the device will lance a suitable body part (e.g., the upper arm) so that a small quantity of blood, <250 may by collected by the device. The blood specimens are then shipped to the laboratory for testing.

The blood samples will be analyzed by the laboratory for Δ⁹-THC, 11-OH-Δ⁹-THC, Δ⁹-THC-COOH, 8β-hydroxy-Δ⁹-THC, 8β,11-dihydroxy-Δ⁹-THC, CBG, CBN, Δ⁹-THCV, Δ⁹-THC-glucuronide, and Δ⁹-THC epoxides using validated analytical methods; for example, high-performance liquid chromatography (HPLC), gas chromatography tandem mass spectrometry (GC-MS/MS) and liquid chromatography tandem mass spectrometry (LC-MS/MS). Once the concentrations of these compounds have been determined, the various Δ⁹-THC and metabolite ratios described above will be calculated so that a determination of the time of last cannabis use can be made.

The described model can be used by itself to assess recent use of cannabis, or it can be used in combination with other technologies, for example, a Δ⁹-THC breathalyzer or urine analysis. The application of this model is not restricted to just a two-point analysis. Three, four or more samples can be collected, for example, in a workplace setting to further strengthen the predictive accuracy of the model. Another advantage of this invention is that it can be used to assess recent use of cannabis edibles, unlike other technologies, for example, oral fluid analysis and Δ⁹-THC breathalyzers, which can be unreliable in this setting.

Yet a further advantage of this invention is in the setting of workplace drug testing in states or countries where recreational and/or medicinal use of cannabis has been legalized. For employers, the described model will better differentiate those employees who have used cannabis recently, thereby posing a potential safety risk, from employees who have used cannabis in the past, but not recently enough to be impaired. This would protect both the employer, who can identify those employees who pose a genuine threat to the business, and employees, who can avoid unjustified termination as a result of legal and responsible cannabis use. In addition to non-DUI investigations related to cannabis use, this invention can also be applied to DUI and non-DUI investigations related to the use of drugs other than cannabis; for example, determining recent use of illegal synthetic cannabinoids, methamphetamines, opiates, benzodiazepines, cocaine, and barbiturates.

Further refinement of the described model can take into account interactions between cannabis and prescription and non-prescription drugs that can lead to a prolongation of the psychoactive effects, and thus potential for impairment, of cannabis. Based on observations, for example, a prolongation of the half-lives of key cannabinoids, a secondary proprietary test for drug interactions is necessary.

A model based on multiple recent use parameters will be used to determine the probability that a suspect recently used cannabis and was driving within the established “impairment window” (within 4-5 hours after consumption). The greater the number of recent use parameters for which the subject is positive, the higher the statistical probability of recent cannabis use. The model is based on the following parameters that have been associated with recent use of cannabis, whether through smoking, vaping or ingestion of cannabis edibles, with one point being given for each parameter in which the suspect's samples are positive. This list is for illustration purposes only. As additional recent use parameters are identified, they may be added to the model.

The presence of Δ⁹-THC-glucuronide, CBG, CBN, Δ⁹-THCV, or Δ⁹-THC epoxides in plasma or whole blood.

A short Δ⁹-THC half-life (<1 hour), indicating distribution phase kinetics.

A short CBN half-life (<1 hour), indicating distribution phase kinetics.

A short 11-OH-Δ⁹-THC half-life (<1 hour), indicating distribution phase kinetics.

A ratio of 11-OH-Δ⁹-THC to Δ⁹-THC that is increasing by at least 25%.

A ratio of Δ⁹-THC-COOH to Δ⁹-THC that is increasing by at least 25%.

A ratio of Δ⁹-THC-COOH to CBN that is increasing by at least 25%.

A ratio of 8β,11-dihydroxy-Δ⁹-THC to Δ⁹-THC that is increasing by at least 25%.

An increasing plasma or whole blood Δ⁹-THC concentration, indicating recent consumption of cannabis edibles.

Approximately equal concentrations of Δ⁹-THC and 11-OH-Δ⁹-THC in plasma or whole blood, indicating recent consumption of cannabis edibles.

Approximately equal concentrations of Δ⁹-THC and Δ⁹-THC-COOH in plasma or whole blood.

A Δ⁹-THC plasma or whole blood concentration>5 ng/mL combined with a Δ⁹-THC-COOH/11-OH-Δ⁹-THC concentration ratio<20.

Consistently higher concentrations of Δ⁹-THC-COOH compared to Δ⁹-THC and 11-OH-Δ⁹-THC, indicating recent consumption of cannabis edibles.

In summary, a pharmacologic model will be developed for the assessment of recent cannabis use. As a theoretical example, a subject who is positive for at least four (4) recent use parameters out of a total of six (6) has an approximately 99% probability of having used cannabis recently, a test result that can be used as further evidence to support a law enforcement officer's determination of driver impairment.

II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a parameter” includes a plurality of such parameters and reference to “the metabolite” includes reference to one or more metabolites known to those skilled in the art, and so forth.

The terms “subject,” “patient,” or “individual” are used herein interchangeably. The term “sample” includes, whole blood, plasma, serum, oral fluid and urine.

As used herein, the term “consumption” includes smoking, vaping, sublingual administration, oral administration, topical contact, and administration as a suppository. One skilled in the art will know of additional methods of administering cannabis and cannabis-derived compounds.

The term “cannabinoid” refers any member of the broad class of phytocannabinoid compounds derived from the cannabis plant (Cannabis spp.), or their synthetic equivalents, including, but not limited to Δ⁸-THC, Δ⁹-THC, CBD, CBG, CBC, Δ⁹-THCV, CBN and any of their associated carboxylic acid forms.

The term “metabolite” includes any of the products resulting from the metabolism of cannabinoids within the body, including but not limited to 11-OH-Δ⁹-THC, 11-nor-9-carboxy-Δ⁹-THC, 8β-hydroxy-Δ⁹-THC, 8β,11-dihydroxy-Δ⁹-THC, Δ⁹-THC-glucuronide, and Δ⁹-THC epoxides.

Δ⁸-THC and Δ⁹-THC refer to, respectively, Δ⁸-tetrahydrocannabinol and Δ⁹-tetrahydrocannabinol.

11-OH-Δ⁹-THC refers to the metabolite 11-hydroxy-Δ⁹-tetrahydrocannabinol.

Δ⁹-THC-COOH refers to the metabolite 11-nor-9-carboxy-Δ⁹-tetrahydrocannabinol.

Δ⁹-THCV refers to the cannabinoid Δ⁹-tetrahydrocannabivarin.

CBN refers to the cannabinoid cannabinol.

CBD refers to the cannabinoid cannabidiol.

CBG refers to the cannabinoid cannabigerol.

CBC refers to the cannabinoid cannabichromene.

The term “illegal synthetic cannabinoid” refers to any of the illegal cannabinoid-like designer drugs that have been identified, including but not limited to, Spice, K2, synthetic marijuana, AK-47, Mr. Happy, Scooby Snax, Kush, and Kronic.

III. Description of the Method

The method relies on the use a statistical model to derive the probability that a subject recently used cannabis or some other substance of abuse for which testing is desired. Comprising the model are parameters that have been associated with the recent use of cannabis or other compounds of interest. At least six parameters are used, but more may be added as they are identified to strengthen the statistical power of the model. The subjects' pharmacokinetic data are used to determine whether the particular subject is positive, for which a value of “1” is assigned or negative, for which a value of “0” is assigned, in each of the parameters. An appropriate statistical test is then applied to determine whether the subjects' average scores are significantly different from the hypothetical average of “0,” which would represent someone who has not used the substance recently.

For example, the following six parameters have been associated with the recent use cannabis: (1) a short Δ⁹-THC half-life (<1 hour), indicating distribution phase kinetics; (2) a short CBN half-life (<1 hour), indicating distribution phase kinetics; (3) a ratio of 11-OH-Δ⁹-THC to Δ⁹-THC that is increasing by at least 25%; (4) a ratio of Δ⁹-THC-COOH to Δ⁹-THC that is increasing by at least 25%; (5) a ratio of Δ⁹-THC-COOH to CBN that is increasing by at least 25%; and (6) a ratio of 8β,11-dihydroxy-Δ⁹-THC to Δ⁹-THC that is increasing by at least 25%. After determining positivity or negativity for each of these six parameters, a Student's t-test with two-sample equal variance, two-tailed distribution, and significance level of 0.05 is then applied. The null hypothesis for this test is that there is no difference between the subject's average for the six parameters and a hypothetical non-recent user. The result of this test will return the probability (p) that the test subject could have come from a population of non-recent users. Thus, a p-value of 0.05 indicates a 5% probability of non-recent use, meaning a 95% probability of recent use of cannabis. A p-value of 0.01 indicates a 1% probability of non-recent use, which translates to a 99% probability of recent use of cannabis. When using a model comprised of six parameters, a subject who is positive for three parameters has a 95% probability of recent use. A subject who is positive for four or more parameters has a 99% probability of recent use. A p-value result of <0.05 is considered statistically significant. Other statistical tests may be suitable and may be employed in the evaluation. This model may be adapted to test for the recent use of other drugs of interest, and additional parameters may be added to this model to strengthen its statistical power.

### IV. EXAMPLES

The following examples are offered to illustrate, but not to limit, the claimed invention.

A total of 20 human subjects were included in a clinical trial designed to evaluate the feasibility of the described method for determining recent use of cannabis. Also evaluated in this trial was the safety and feasibility of an experimental blood draw device, compared to a standard lancet device, that automatically collects and stores whole blood in a tube containing anticoagulant. Subjects were asked to describe their experience with both types of blood draw devices. Blood samples were collected prior to smoking and then at various time points up to 200 minutes post-smoking, focusing on three major time intervals: (1) 0 to 20 minutes post-smoking; (2) 120-140 minutes post-smoking; and (3) 180-200 minutes post-smoking. For cannabis edibles, blood samples were collected up to eight hours after ingestion.

### Example 1

### Determining Recent Use of Cannabis—Immediately After Smoking to 20 Minutes Post-Smoking

The goal of examining the 20-minute period immediately after smoking was to assess the ability of the described model to detect recent use of cannabis when the evidence of recent use should be at or near maximal levels.

A total of 14 subjects had blood samples collected immediately after smoking and 20 minutes post-smoking. Following written informed consent, subjects were given a cannabis cigarette to smoke that contained approximately 500 mg of a high-potency cannabis strain. Subjects were instructed to smoke the entire cigarette, or as much as they could, within a 10-minute period. Blood samples were collected using a either a standard lancet device (13 subjects) or the experimental blood draw device described above (1 subject). Blood samples were then extracted and analyzed for Δ⁹-THC, Δ⁹-THC metabolites, and other cannabinoids by liquid chromatography tandem mass spectrometry (LC-MS/MS). Using the results, the following six parameters were then calculated: Δ⁹-THC half-life; CBN half-life; 11-OH-Δ⁹-THC/Δ⁹-THC ratio; Δ⁹-THC-COOH/Δ⁹-THC ratio; Δ⁹-THC-COOH/CBN ratio; 8β,11-dihydroxy-Δ⁹-THC/Δ⁹-THC ratio. These six parameters were also computed for the 11 subjects from whom baseline blood samples were collected 20 minutes apart prior to smoking.

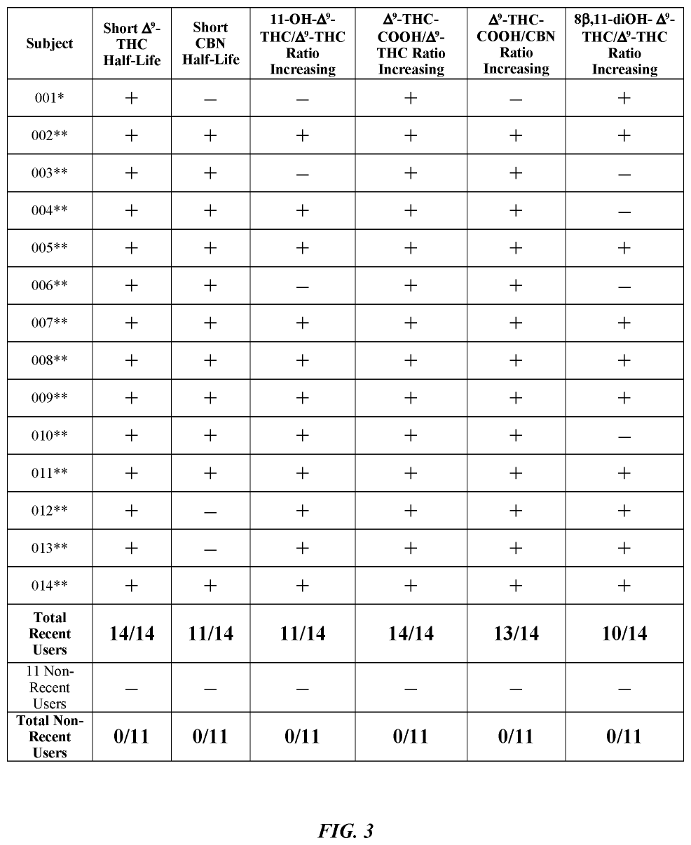

FIG. 3 shows the number of parameters associated with recent use of cannabis for which each subject was positive or negative. All 14 subjects showed statistically significant evidence (p<0.01 in 13 subjects; p<0.05 in 1 subject) of recent cannabis use based on the described model, while none of the blood samples collected from 11 subjects prior to smoking showed evidence of recent use.

### Example 2

### Determining Recent Use of Cannabis—120 to 140 Minutes Post-Smoking

The goal of examining the time period approximately two hours after smoking was to evaluate the ability of the described model to predict recent use of cannabis in a more realistic setting, where someone smokes cannabis, decides to drive about an hour later, and is then tested for suspicion of DUI about two hours after having smoked.

A total of six subjects had blood samples collected at 120 minutes and 140 minutes post-smoking. Following written informed consent, subjects were given a cannabis cigarette to smoke that contained approximately 500 mg of a high-potency cannabis strain. Subjects were instructed to smoke the entire cigarette, or as much as they could, within a 10-minute period. Blood samples were collected using a either a standard lancet device (5 subjects) or the experimental blood draw device described above (1 subject). Blood samples were then extracted and analyzed for Δ⁹-THC, Δ⁹-THC metabolites, and other cannabinoids by LC-MS/MS. Using the results, the following six parameters were then calculated: Δ⁹-THC half-life; CBN half-life; 11-OH-Δ⁹-THC/Δ⁹-THC ratio; Δ⁹-THC-COOH/Δ⁹-THC ratio; Δ⁹-THC-COOH/CBN ratio; 8β,11-dihydroxy-Δ⁹-THC/Δ⁹-THC ratio. These six parameters were also computed for the 11 subjects from whom baseline blood samples were collected 20 minutes apart prior to smoking.

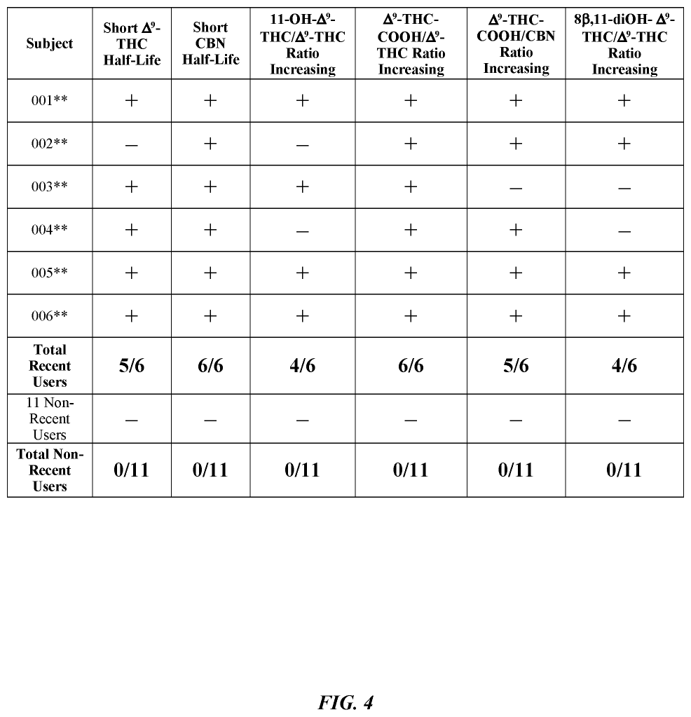

FIG. 4 shows the number of parameters associated with recent use of cannabis for which each subject was positive or negative. All six subjects were positive for at least four recent use parameters, which was statistically significant evidence (p<0.01, all subjects) of recent cannabis use based on the described model, while none of the blood samples collected from 11 subjects during a 20-minute interval prior to smoking showed evidence of recent use.

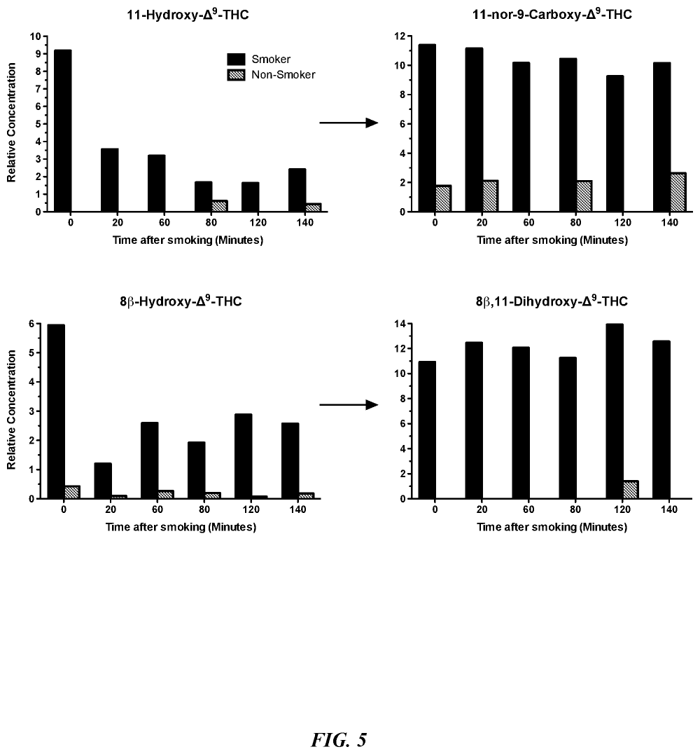

FIG. 5 shows the conversion of 11-OH-Δ⁹-THC to 11-nor-9-carboxy-Δ⁹-THC and the conversion of 8β-hydroxy-Δ⁹-THC to 8β,11-dihydroxy-Δ⁹-THC in a recent cannabis smoker compared to an non-recent smoker up to 140 minutes post-smoking. The relative levels of 11-nor-9-carboxy-Δ⁹-THC and 8β,11-dihydroxy-Δ⁹-THC are clearly increasing in the smoker, while they remain relatively unchanged in the non-recent smoker.

### Example 3

### Determining Recent Use of Cannabis—180 to 200 Minutes Post-Smoking

The goal of evaluating the time period approximately three hours after smoking was to evaluate the ability of the model to predict recent use when pharmacologic evidence of such should be waning, but impairment is still possible.

A total of eight subjects had blood samples collected at 180 minutes and 200 minutes post-smoking. Following written informed consent, subjects were given a cannabis cigarette to smoke that contained approximately 500 mg of a high-potency cannabis strain. Subjects were instructed to smoke the entire cigarette, or as much as they could, within a 10-minute period. Blood samples were collected using a either a standard lancet device (7 subjects) or the experimental blood draw device described above (1 subject). Blood samples were then extracted and analyzed for Δ⁹-THC, Δ⁹-THC metabolites, and other cannabinoids by LC-MS/MS. Using the results, the following six parameters were then calculated: Δ⁹-THC half-life; CBN half-life; 11-OH-Δ⁹-THC/Δ⁹-THC ratio; Δ⁹-THC-COOH/Δ⁹-THC ratio; Δ⁹-THC-COOH/CBN ratio; 8β,11-dihydroxy-Δ⁹-THC/Δ⁹-THC ratio. These six parameters were also computed for the 11 subjects from whom baseline blood samples were collected 20 minutes apart prior to smoking.

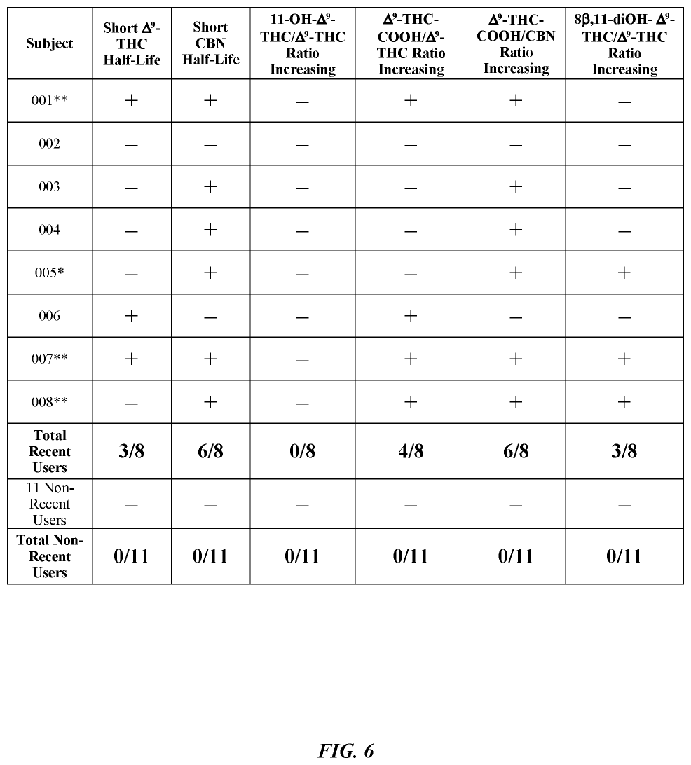

FIG. 6 shows the number of parameters associated with recent use of cannabis for which each subject was positive or negative. At this time interval post-smoking, only four of the eight subjects showed statistically significant evidence of recent cannabis use (p<0.01 in three subjects; p<0.05 in one subject) based on the described model. None of the blood samples collected from 11 subjects collected over a 20-minute interval prior to smoking showed evidence of recent use.

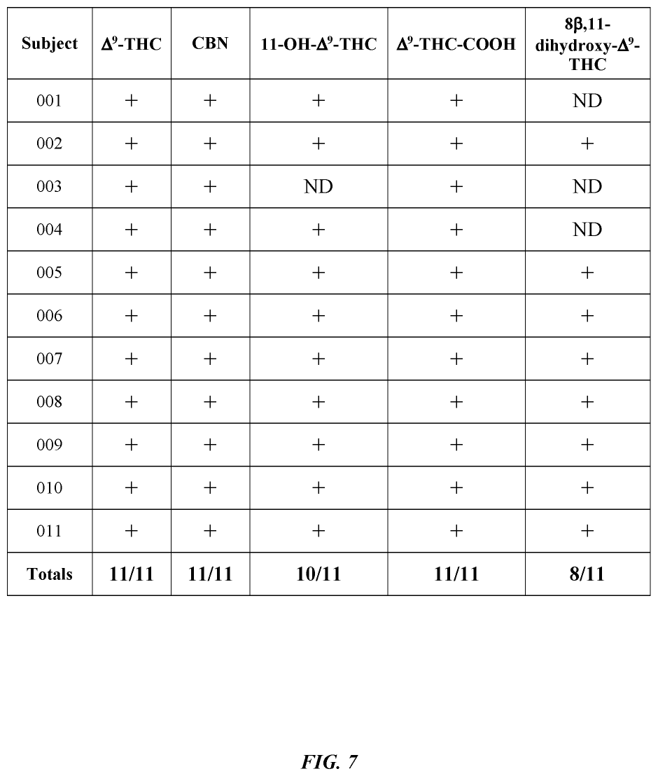

FIG. 7 shows that Δ⁹-THC, CBN and the three Δ⁹-THC metabolites included in the parameters used to determine recent use in these examples were still detected in the majority of the 11 subjects from whom blood samples were collected during a 20-minute interval prior to smoking; however, when subjected to the recent use parameters defined in these examples, the data did not meet the established criteria in any subject.

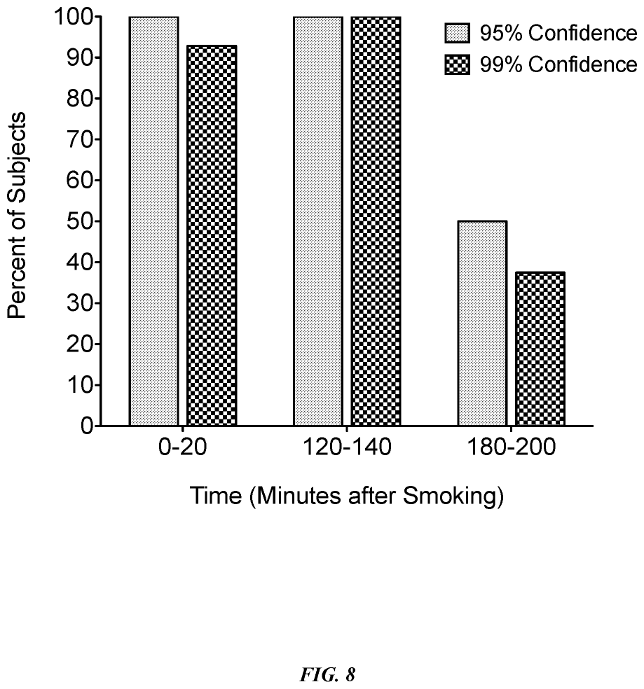

FIG. 8 shows the degree of confidence in predicting recent use of cannabis in the three time intervals evaluated post-smoking. Within two hours post-smoking, the described model predicted recent use in all subjects with at least 95% confidence, and in most subjects with at least 99% confidence. At three hours post-smoking, the model was capable of predicting recent use in half of the subjects with at least 95% confidence.

The results from these three examples demonstrate that the described model accurately determines recent use of cannabis within approximately two hours post-smoking and is still about 50% effective in identifying recent use within three hours post-smoking. As shown in FIG. 7, the key cannabinoids and metabolites included in the parameters used to determine recent use in these examples were still detected in almost all of the 11 subjects prior to smoking. This is an important point because non-recent cannabis users will show detectable levels of several or all the compounds evaluated in these studies. An effective test for the recent use of cannabis must be able to discriminate between past use and recent use, which carries the risk of impairment. The results presented in these examples demonstrate that the described model can successfully determine recent use of cannabis within a background of non-recent use.

### Example 4

### Determining Recent Use of Cannabis—Smoking versus Edibles Case Study

The goal of this case study was to assess the value of the model in predicting recent use in the setting of cannabis edibles, the popularity of which is increasing and the impairment from which is delayed compared to smoking.

As a clinical case study, a subject was studied to determine recent use of cannabis following ingestion of a cannabis-extract infused chocolate cannabis edible containing 30 mg of Δ⁹-THC compared to smoking a cannabis cigarette containing approximately 500 mg of high-potency cannabis. Following written informed consent, two baseline blood samples were collected 30 minutes apart using a lancet device. The subject was then given three cannabis edibles to eat, each containing 10 mg of Δ⁹-THC. Blood samples were then collected every 30 minutes up to eight hours post-administration using a lancet device. After extraction and analysis, the following six parameters were then calculated: Δ⁹-THC half-life; CBN half-life; 11-OH-Δ⁹-THC/Δ⁹-THC ratio; Δ⁹-THC-COOH/Δ⁹-THC ratio; Δ⁹-THC-COOH/CBN ratio; 8β,11-dihydroxy-Δ⁹-THC/Δ⁹-THC ratio. The results were compared to those obtained after the subject smoked a cannabis cigarette in a previous study.

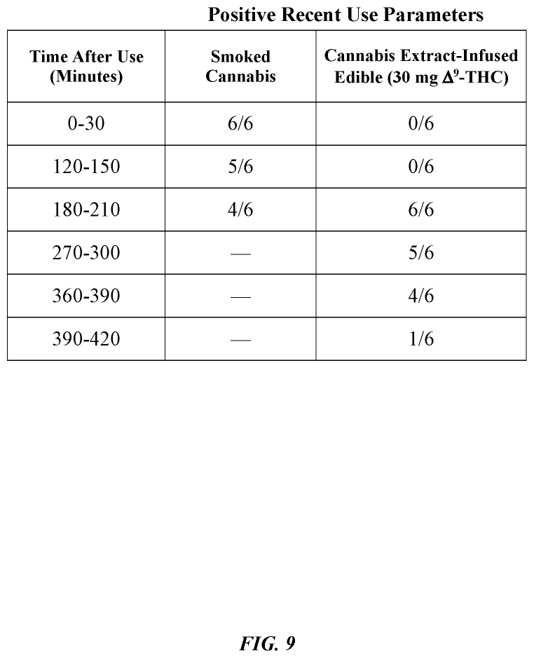

FIG. 9 shows the number of parameters associated with recent use of cannabis for which this subject was positive or negative at each time interval after smoking or consuming a cannabis edible. While convincing evidence of recent use appeared within the first 30 minutes after smoking, no such evidence was observed until three hours after the subject had consumed the cannabis edible. This is consistent with published data regarding the delayed absorption of cannabinoids following edible cannabis administration compared to smoking. Importantly, this subject displayed an identical pattern of positive recent use parameters between three and six hours after consuming the cannabis edible compared to the first three hours after smoking.

The results of this case study demonstrate that the described model can also be used to determine recent use of cannabis edibles, which may prove valuable given that use of edible cannabis products is increasing. Due to the comparatively very low levels of Δ⁹-THC following ingestion of edibles compared to smoking, Δ⁹-THC breathalyzers may prove unreliable, besides the fact that using Δ⁹-THC alone to assess recent use is problematic at best. While oral fluid analysis may be useful in determining recent use within the past 24 hours, it cannot be used to determine impairment. Furthermore, due to contamination of the oral cavity, saliva or oral fluid analysis is unreliable for determining recent use of cannabis following consumption of edibles.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method for determining recent use of cannabis in a subject, the method comprising collecting two or more whole blood samples separated in time by approximately 20 to 30 minutes, analyzing them for Δ⁹-THC, Δ⁹-THC metabolites, and other cannabinoids, computing specific pharmacokinetic parameters, and then evaluating the results based on the criteria of at least six parameters associated with the recent use of cannabis.

2. The method of claim 1, whereby a subject is evaluated by law enforcement personnel for driving under the influence (DUI) of cannabis by collecting two or more whole blood samples.

3. The method of claim 2, whereby the blood samples are collected using a device that automatically collects and stores capillary blood for later laboratory analysis.

4. The method of claim 3, whereby blood sample collection is performed in combination with other cannabis recent use detection technologies such as a Δ⁹-THC breathalyzer or oral fluid analysis device.

5. The method of claim 1, whereby an employee or prospective employee is tested for recent use of cannabis by an employer or prospective employer.

6. The method of claim 5, whereby the subject is tested for recent cannabis use by collecting two or more whole blood samples using a device that automatically collects and stores capillary blood for later laboratory analysis.

7. The method of claim 5, whereby the subject is tested for recent cannabis use by collecting two or more whole blood samples using a lancet or other means of blood collection.

8. The method of claim 5, whereby the subject is tested for recent cannabis use by collecting two or more samples of oral fluid or urine in addition to whole blood.

9. The method of any one of claims 2 through 8, whereby the samples collected are also analyzed for prescription and non-prescription drugs, including alcohol, benzodiazepines, barbiturates, opiates, tricyclic antidepressants, selective serotonin reuptake inhibitors, and antifungals.

10. A kit comprising blood collection devices, sample tubes, gloves, sample tube labels, and sample shipping container.

11. The kit of claim 10, wherein the blood collection devices automatically collect and store capillary blood for later laboratory analysis.

12. The kit of claim 10, wherein the blood collection devices are sterile, disposable lancets.

13. The method of claim 1, whereby the parameters used for determining recent use of cannabis are specifically adapted for cannabis edibles.

14. The method of any one of claims 2 through 8, whereby the samples are analyzed and assessed using recent use parameters specific for cannabis edibles.

15. A method for determining recent use of other drugs of abuse, for example, illegal synthetic cannabinoids, methamphetamine, opiates, benzodiazepines, barbiturates, and cocaine, the method comprising collecting two or more whole blood samples separated in time by approximately 20 to 30 minutes, analyzing them for key drug molecules and metabolites, computing specific pharmacokinetic parameters, and then evaluating the results based on the criteria of at least six parameters associated with the recent use of each respective drug compound.

16. The method of claim 15, whereby a subject is evaluated by law enforcement personnel for driving under the influence (DUI) of drugs by collecting two or more whole blood samples.

17. The method of claim 16, whereby the blood samples are collected using a device that automatically collects and stores capillary blood for later laboratory analysis.

18. The method of claim 15, whereby an employee or prospective employee is tested for recent use of drugs by an employer or prospective employer.

19. The method of claim 18, whereby the subject is tested for recent drug use by collecting two or more whole blood samples using a device that automatically collects and stores capillary blood for later laboratory analysis.

20. The method of claim 18, whereby the subject is tested for recent drug use by collecting two or more whole blood samples using a lancet or other means of blood collection.

21. The method of claim 18, whereby the subject is tested for recent drug use by collecting two or more samples of oral fluid or urine in addition to whole blood.
